(19) United States
(12) Patent Application Publication
(10) Pub. No.: US 2010/0274576 A1
(43) Pub. Date: Oct. 28, 2010

(54) SYSTEMS AND METHODS FOR ENHANCING THE TREATMENT OF INDIVIDUALS

(75) Inventor: Robert Jay Young, Media, PA (US)

Correspondence Address: Robert Jay Young, 1556 Elgin Court, Keswick, VA, 22947, US

(73) Assignee: Robert Jay Young, Pittsburgh, PA (US)

(21) Appl. No.: 12/636,400

(22) Filed: Dec. 11, 2009

Related U.S. Application Data
(60) Provisional application No. 61/121,542, filed on Dec. 11, 2008.

(51) Int. Cl. G06Q 50/00; A61B 5/00

(52) U.S. Cl. 705/2; 600/300

(57) ABSTRACT

Methods and systems of managing treatment for a patient are disclosed. Data pertaining to an intervention may be received by a processor. The intervention may include one or more of a homework intervention, an in-session intervention, or a combination thereof. The data pertaining to the first intervention may be stored in a database. The processor may transmit at least a portion of the data pertaining to the intervention to a patient computer. The processor may receive feedback information from the patient computer pertaining to the intervention. The feedback information may be stored in the database in association with the data pertaining to the first intervention. The feedback information may then be displayed to a treatment provider.

### CROSS-REFERENCE TO RELATED APPLICATION

This application claims priority to U.S. Provisional Application No. 61/121,542, filed Dec. 11, 2008 and entitled “PSYCAIDE: A Web-Based Application Designed to Enhance the Use and Effectiveness of Therapeutic Homework,” which is incorporated herein by reference in its entirety.

### BACKGROUND

Health care providers can provide a variety of healthcare services to patients. For example, treatment is traditionally provided in a doctor's office or a hospital setting. However, treatment can also be performed outside remote from such locations as well, such as at the patient's residence or independent of, but under the direction of, a health care provider. In some case, treatment provided remotely from a traditional setting could have reduced effectiveness because remote health care providers may receive less training and/or have fewer therapeutic skills than those in a traditional healthcare environment, because it is difficult to collect accurate information from a patient between sessions, because a structured treatment regimen may not be used and/or the like.

In addition, treatment non-compliance by patients can also hinder the effectiveness of therapeutic homework. For example, a patient could be unaware of how to comply with medication requirements, treatment regimens and the like. As such, the patient might not attempt to administer the treatment, might attempt to administer the treatment incorrectly or incompletely, and/or the like. Moreover, homework interventions are often not crafted to fit an individual patient's needs, abilities, circumstances, and/or the like.

In some cases, primary care physicians may be unable to assist a patient as well. For example, physicians might not be trained to provide therapy, continuously monitor their patients' symptoms, medication compliance and treatment effects, or commit the time necessary to treat specific types of disorders, such as psychiatric disorders. In addition, if care is provided by multiple health care providers, such as a physician and a psychiatrist, it may be difficult to achieve close collaboration between such health care providers due to schedule conflicts, physical distance and the like.

### SUMMARY

Before the present systems, devices and methods are described, it is to be understood that this disclosure is not limited to the particular systems, devices and methods described, as these may vary. It is also to be understood that the terminology used in the description is for the purpose of describing the particular versions or embodiments only, and is not intended to limit the scope.

It must also be noted that as used herein and in the appended claims, the singular forms “a,” “an,” and “the” include plural references unless the context clearly dictates otherwise. Thus, for example, reference to a “device” is a reference to one or more devices and equivalents thereof known to those skilled in the art, and so forth. Unless defined otherwise, all technical and scientific terms used herein have the same meanings as commonly understood by one of ordinary skill in the art. Although any methods, materials, and devices similar or equivalent to those described herein can be used in the practice or testing of embodiments, the preferred methods, materials, and devices are now described. All publications mentioned herein are incorporated by reference. Nothing herein is to be construed as an admission that the embodiments described herein are not entitled to antedate such disclosure by virtue of prior invention. As used herein, the term “comprising” means “including, but not limited to.”

In an embodiment, a computer-implemented system for managing treatment for a patient may include a processor, a processor-readable storage medium in communication with the processor, and a communication interface in communication with the processor. The processor-readable storage medium may contain one or more programming instructions for receiving, by the processor via the communication interface, data pertaining to an intervention comprising one or more of a homework intervention, an in-session intervention, and a combination intervention, storing the data pertaining to the intervention, transmitting, via the communication interface, at least a portion of the data pertaining to the intervention to a patient computer, receiving feedback information from the patient computer via the communication interface, storing the feedback information in association with the data pertaining to the intervention, and causing, via the processor, the feedback information to be transmitted to a treatment provider computer.

In an embodiment, a method of managing treatment for a patient may include receiving, by a processor, data pertaining to a first intervention comprising one or more of a homework intervention, an in-session intervention, and a combination intervention, storing the data pertaining to the first intervention in a database, transmitting, via the processor, at least a portion of the data pertaining to the first intervention to a patient computer, receiving, by the processor, feedback information from the patient computer, storing the feedback information in the database in association with the data pertaining to the first intervention, and displaying the feedback information to a treatment provider.

In an embodiment, a method of managing collaborative treatment for a patient may include receiving, by a processor, data pertaining to a first intervention comprising one or more of a homework intervention, an in-session intervention, and a combination intervention, storing the data pertaining to the first intervention in a database, transmitting, via the processor, at least a portion of the data pertaining to the first intervention to a patient computer, receiving, by the processor, feedback information from the patient computer, storing the feedback information in the database in association with the data pertaining to the first intervention, and enabling access to the data pertaining to the first intervention and the feedback information to a plurality of treatment providers.

### BRIEF DESCRIPTION OF THE DRAWINGS

Aspects, features, benefits and advantages of the present invention will be apparent with regard to the following description and accompanying drawings, of which:

FIG. 1 depicts an exemplary graphical user interface screen for creating a professional user account according to an embodiment.

FIG. 2 depicts an exemplary graphical user interface for a treatment provider account according to an embodiment.

FIG. 3 depicts a flow diagram of a method for creating a patient account and assigning therapeutic homework according to an embodiment.

FIG. 4 depicts an exemplary graphical user interface screen for creating a patient account according to an embodiment.

FIG. 5 depicts an exemplary graphical user interface screen for an initial clinical interview according to an embodiment.

FIG. 6 depicts an exemplary graphical user interface screen for providing a diagnosis for a patient according to an embodiment.

FIG. 7 depicts an exemplary graphical user interface screen for selecting a treatment approach for a patient according to an embodiment.

FIGS. 8a-8d depict exemplary graphical user interface screens for assisting a treatment provider with treating a patient according to an embodiment.

FIG. 9a depicts an exemplary graphical user interface screen for specifying a homework intervention according to an embodiment.

FIG. 9b depicts an exemplary graphical user interface screen for receiving information for a homework intervention according to an embodiment.

FIG. 10 depicts an exemplary graphical user interface screen for specifying and conducting an in-session intervention according to an embodiment.

FIG. 11 depicts a flow diagram of an exemplary method for accessing a homework intervention by a patient according to an embodiment.

FIG. 12 depicts an exemplary graphical user interface screen for a patient to receive and record information pertaining to homework interventions according to an embodiment.

FIG. 13 depicts an exemplary graphical user interface screen for a patient to receive and record information pertaining to a particular homework intervention according to an embodiment.

FIG. 14 depicts an exemplary graphical user interface screen used to review data recorded from one or more interventions according to an embodiment.

FIG. 15 depicts an exemplary supervisor graphical user interface screen according to an embodiment.

FIG. 16 depicts an exemplary researcher graphical user interface screen according to an embodiment.

FIG. 17 depicts a block diagram of exemplary internal hardware that may be used to contain or implement program instructions according to an embodiment.

FIG. 18 depicts a block diagram of an exemplary computer network according to an embodiment.

### DETAILED DESCRIPTION

The following terms shall have, for the purposes of this application, the respective meanings set forth below.

A “treatment provider” refers to a healthcare professional who directly treats patients. A treatment provider may refer to, without limitation, a psychologist, a psychotherapist, a social worker, a physician and/or the like. In a school-based embodiment, a treatment provider may include, without limitation, teachers, guidance counselors, school psychologists, school disciplinarians, principals, assistant principals, coaches, parents or guardians and/or the like. In a wraparound embodiment, a treatment provider may include, without limitation, a behavior specialist consultant, a mobile therapist, a therapeutic staff support, a therapeutic staff support assistant, and/or the like.

As used herein, “homework” or “therapeutic homework” refers to any activity that a patient attempts in the time between therapy sessions or visits to one or more treatment providers that furthers the goals of the therapy. Homework can enable, for example, a mental and/or behavioral health patient to take an active role in his treatment by engaging in activities related to the treatment goals during the time period between therapy sessions and in the absence of direct therapeutic support or therapeutic support provided by minimally trained providers. Homework can also be used by physicians to encourage medication compliance, regular exercise, healthy eating habits, smoking cessation and/or the like. Exemplary homework activities may include, without limitation, regular exercise, taking note of maladaptive automatic thoughts and learning to question their validity, reading educational materials, monitoring one's own symptoms, behaviors and thinking patterns, exposure to stimuli to reduce physiological reactions to anxiety or stress triggers, learning to become used to innocuous bodily sensations, becoming involved in pleasurable activities, testing maladaptive beliefs and predictions, implementing solutions, and practicing, reinforcing and generalizing skills learned in therapy.

A “homework intervention” refers to an intervention or activity assigned to a patient that is intended to be completed by the patient outside of a therapy session or visit to a treatment provider. A treatment provider may assign a homework intervention to a patient using the system described herein in order to treat one or more symptoms, disorders, illnesses and/or the like.

A “supervisor” refers to a healthcare professional that directly or indirectly manages the activities of treatment providers. For example, a licensed supervisor may

A “researcher” refers to an individual that is managing a study based on information retrieved from a plurality of patients. The researcher may collect data from patients being treated by a plurality of treatment providers and/or at a plurality of treatment sites or may administer a study designed to collect such data.

“Collaborative care providers” refer to two or more treatment providers that are providing treatment to the same patient in concert. For example, a psychologist and a physician each providing treatment to the same patient may be considered to be collaborative care providers. In another example, a team of treatment providers having similar skill sets may perform different tasks in different settings with a given patient.

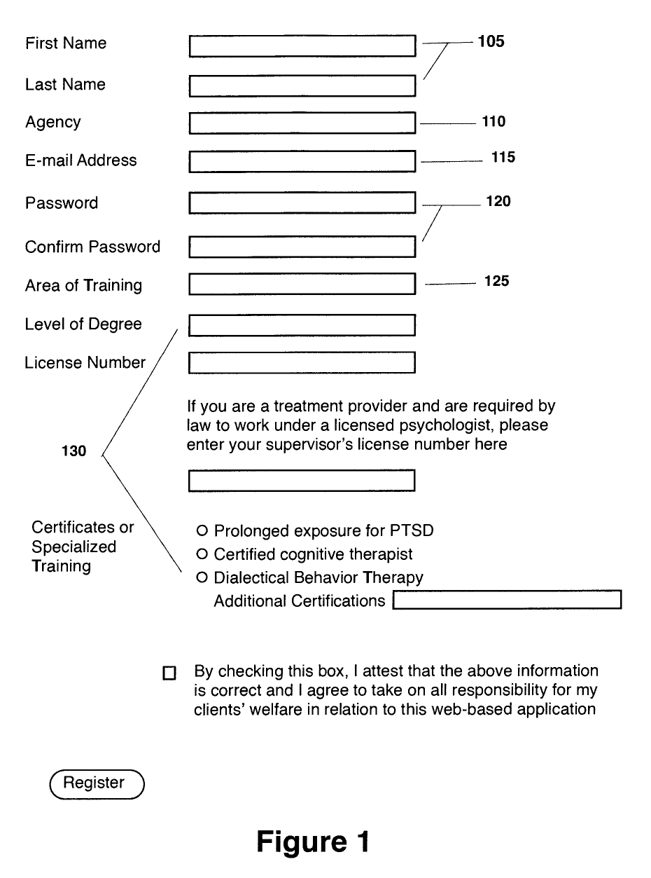

FIG. 1 depicts an exemplary graphical user interface screen for creating a professional user account according to an embodiment. As shown in FIG. 1, a healthcare professional, such as a treatment provider, a clinical supervisor, a researcher and/or the like can create an account on the system. The professional user may enter identifying information, such as their name 105, agency or affiliation 110, email address 115, password 120, training area 125, and professional credentials 130. Different professional users may gain access to different portions of the system based on the information that is provided. In an embodiment, an administrator may review the entered material in order to assure that the account requester is a healthcare professional or other qualified or acceptable user. Upon approval, the user may be informed that the account is active.

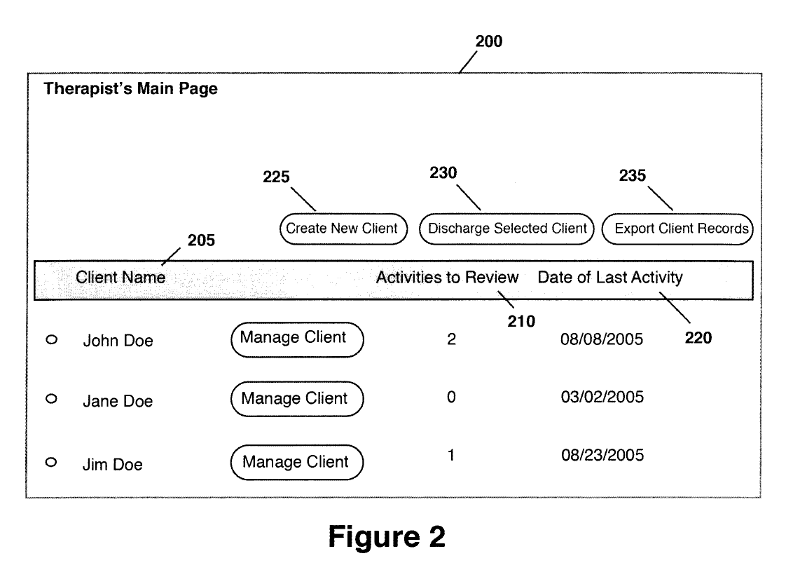

FIG. 2 depicts an exemplary graphical user interface for a treatment provider account according to an embodiment. As shown in FIG. 2, a treatment provider may be redirected to a treatment provider screen 200 upon login. The treatment provider screen 200 may provide information to the treatment provider regarding the treatment and intervention status for patients being treated by the treatment provider. The treatment provider screen 200 may include, for example, a list of existing clients 205, a number of activities to review for each client 210, and a date of the last activity 220 by the client within the system. In addition, the treatment provider screen 200 may include a selection to enable new clients to be added 225 to the database or deleted 230 from the list. In an embodiment, if a client is deleted 230 from the list, the data for such client may still be stored permanently as a electronic medical record. Records for one or more patients being treated by the treatment provider may also be exported 235 for transfer to a different computer system. The process for adding 225 a new client to the system is described in further detail below. As used herein, the terms “patient” and “client” are used interchangeably to refer to an individual designated to receive treatment.

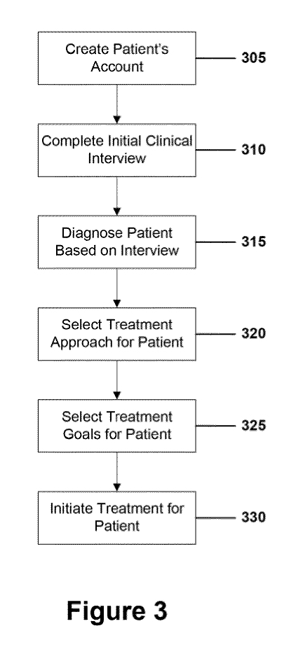

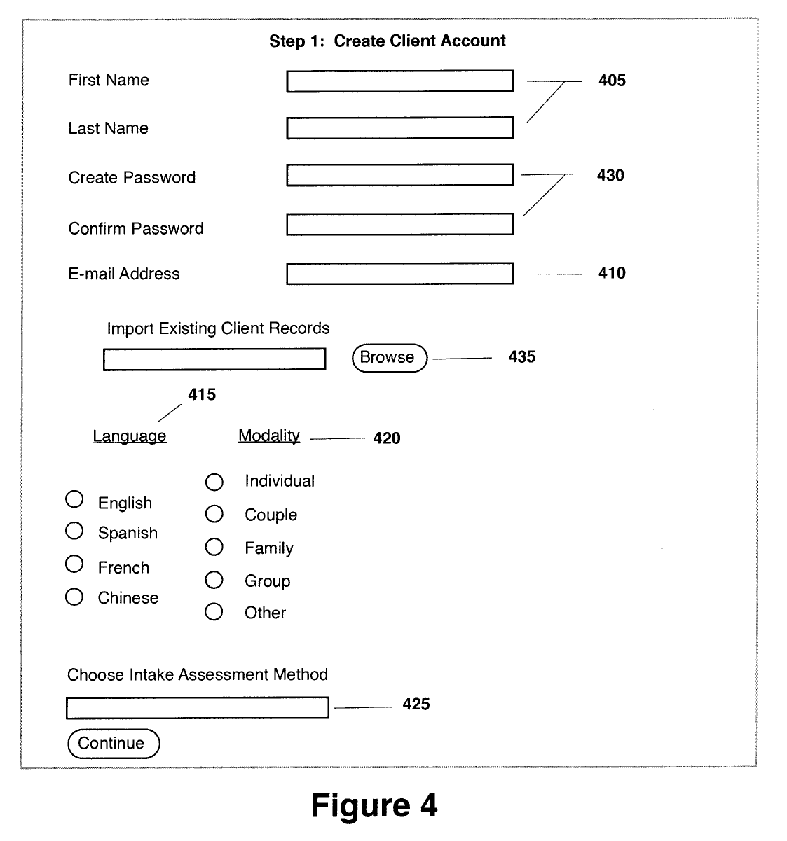

FIG. 3 depicts a flow diagram of a method for creating a patient account and assigning therapeutic homework according to an embodiment. As shown in FIG. 3, a treatment provider may create 305 a patient's account. In an embodiment, the treatment provider may create 305 the account for the patient by accessing an option within the treatment provider's account. As such, a patient account may be created 305 as a sub-account of the treatment provider account according to the disclosed embodiment. In an embodiment, individuals that are not seeing a treatment provider may not be able to create 305 an account or use the system as a self-help resource in order to prevent misuse and decrease liability risk for using the system. The patient account may enable the patient to access the system at a location remote from the treatment provider's office in order to access homework interventions. In addition, the patient account may enable progress for the patient to be recorded to enable the patient to track their progress through therapy. An exemplary graphical user interface for creating a patient account is shown in FIG. 4. Other methods of creating a patient account may also be performed within the scope of this disclosure.

When creating 305 a client account, the treatment provider may provide patient information, such as the patient's name 405, email address 410, home address (not shown), a language in which information is displayed 415, a modality by which the patient is treated 420, and whether the initial assessment of the patient is conducted in-person or remotely 425, such as via a connection with a communication device operated by the patient. In an embodiment, the modality by which the patient is treated 420 may include one or more of the following: individual treatment, treatment as a couple, treatment as a group, treatment as a family, and wraparound treatment. Alternate modalities may also be used within the scope of this disclosure. If the initial assessment is performed remotely, the assessment may be performed, for example and without limitation, via an Internet connection with a computer that is accessible to the patient or via a conversation with the patient over a phone connection in which data is entered by an assessor.

In an embodiment, a password 430 may be assigned to the account when the account is being created 305. In an embodiment, the patient or a representative of the patient may select a password 430 for the account. In an alternate embodiment, the treatment provider may select an initial password 430 on behalf of the patient that can be changed at a later time by the patient. Such an embodiment may be used particularly when the patient provides information for the account from a remote location.

In an embodiment, the treatment provider may opt to import existing patient records 435 when creating 305 the patient account. The patient records may include information prepared by one or more previous or concurrent treatment providers, the patient, one or more third parties, one or more health care providers, one or more biological sensors and/or the like.

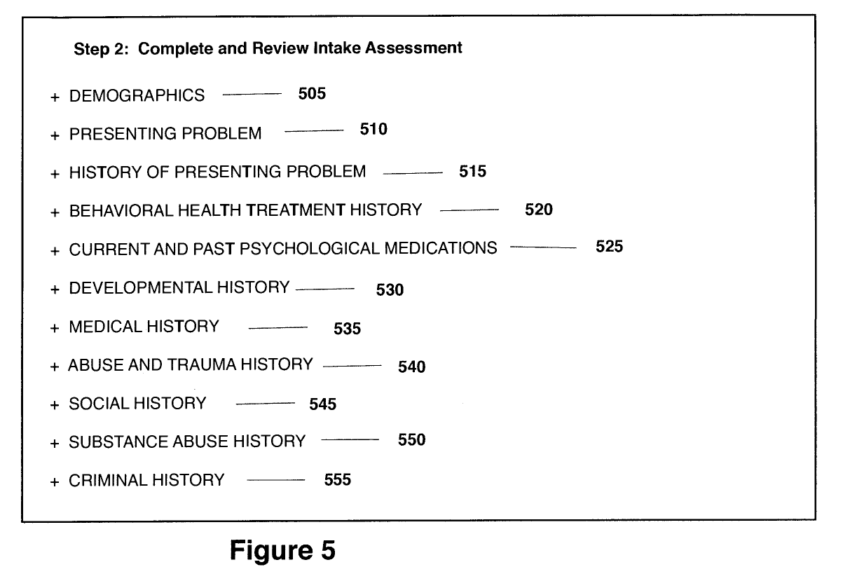

The patient and/or the treatment provider may access and complete 310 an initial clinical interview (or intake assessment). The initial clinical interview may receive substantially comprehensive input regarding certain biological, psychological, social characteristics and/or the like of the patient. Various question categories may be presented depending on the type of treatment to be provided. For example and without limitation, an initial clinical interview for a mental health patient may include question categories pertaining to the patient's demographical information 505, current presenting problems (i.e., symptoms) 510, historical presenting problems 515, behavioral health treatment history 520, current and past psychological medications 525, developmental history 530, medical history 535, history of abuse or trauma 540, social history 545, substance abuse history 550, criminal history 555 and/or the like, as shown in FIG. 5. Selecting a question category header may cause a series of questions to be displayed to a user. If the patient is present during the account creation process, the treatment provider may access and complete 310 the initial clinical interview after creating the account. In an alternate embodiment, if the patient completes the account creation process remotely, the treatment provider may be directed to access and complete 310 the initial clinical interview when the patient first visits the treatment provider's office. In yet another embodiment, the patient may access and complete 310 the initial clinical interview at a location remote from the treatment provider's office. In such an embodiment, an email containing a link to the initial clinical interview may be automatically or manually generated and sent to the patient to enable the patient to access and complete 310 the interview from a remote location. Alternately, the patient may receive a URL address, user name, password and/or the like to access the account over the phone, in person, by mail, and/or the like. Regardless of the manner in which the interview is conducted, the information from the interview may be associated with the patient account and stored in a database when entered.

In an embodiment, answers to particular questions may trigger warning messages to be displayed to the treatment provider. For example, an answer of “yes” to a question “have you ever tried to take your life?” may warrant continued monitoring of potential suicide by the treatment provider.

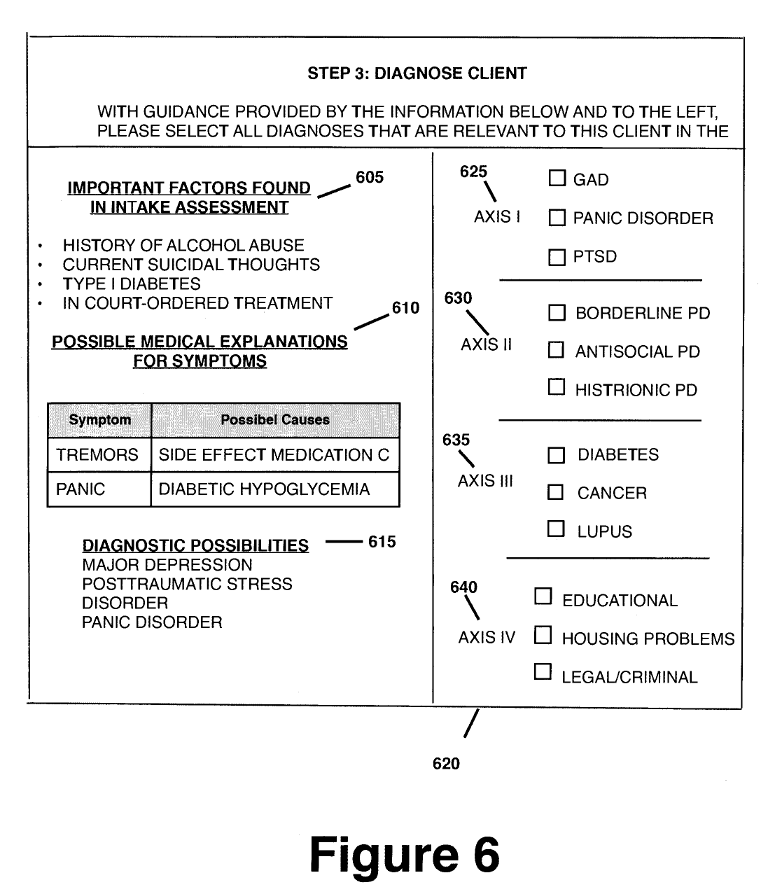

The treatment provider may then diagnose 315 the patient based at least in part on the information received from the initial clinical interview. A diagnosis graphical user interface screen is shown in FIG. 6. The diagnosis screen may include one or more important factors 605 identified from the initial clinical interview, one or more possible medical explanations 610 for symptoms identified by the patient, a list of potential problems 615, and a problem selection section 620. The important factors 605 may correspond to particular issues highlighted during the initial clinical interview or during the course of treatment (i.e., via homework, in-session interventions and/or the like). Displaying such factors 605 may reduce the chance that a treatment provider misses information that is critical to the diagnosis, treatment planning and/or safety of the patient and may also reduce the risk of legal and ethical liability for the treatment provider. The medical explanations 610 may list symptoms identified by the patient during the initial clinical interview or during the course of the treatment and one or more possible physiological causes for each symptom. The causes may include, without limitation, medication side effects, effects of substance abuse, diseases and/or medical conditions, and/or the like. In addition, a list of potential problems 615 may be automatically generated for review by the treatment provider.

The problem selection section 620 may enable the treatment provider to select one or more problems for a patient based on the other information provided by the diagnosis graphical user interface and the treatment provider's experiences with the patient. The one or more selected problems for a patient may determine the treatment approaches and interventions that the system recommends for the patient. In an embodiment, determination of treatment approaches and interventions may be based on information provided by statistical calculations, Monte Carlo simulations, other expert system-related processes, such as forward- and backward-chaining, decision trees, pattern recognition, algorithms and/or the like.

The categorization of diagnoses may depend on type of treatment provided. For example, if the patient requires mental health treatment, the diagnoses may be categorized based on a five axis system of classifying problems that are commonly treated or encountered in the mental health and behavioral health fields. For example, Axis 1 625 may include clinical psychiatric disorders, Axis 2 630 may include mental retardation and personality disorders, Axis 3 635 may include general medical disorders, syndromes and diseases, Axis 4 640 may include psychosocial and environmental problems, and Axis 5 (not shown) may refer to a numerical rating of a patient's general ability to function. In an embodiment, Axis 1 625 may permit a treatment provider to select from clinical psychiatric disorders including generalized anxiety disorder, panic disorder, post-traumatic stress disorder, depression, bipolar disorder, schizophrenia, schizoaffective disorder and/or the like. In an embodiment used in a scholastic environment, Axis 1 625 may enable selection from a list of problems including, without limitation, attention deficit hyperactivity disorder, oppositional defiant disorder, autism, Aspergers disorder, and/or the like. In an embodiment used in a wraparound environment, Axis 1 625 may enable selection from a list of problems including, without limitation, autism, Aspergers disorder, conduct disorder, oppositional defiant disorder, attention deficit hyperactivity disorder, reactive attachment disorder, and/or the like.

In an embodiment, Axis 2 630 may permit a treatment provider to select from disorders including , mental retardation, paranoid personality disorder, schizoid personality disorder, schizotypal personality disorder, antisocial personality disorder, borderline personality disorder, histrionic personality disorder, narcissistic personality disorder, and/or the like.

Axis 3 635 and Axis 4 640 may similarly include a plurality of corresponding disorders, syndromes, diseases, problems and/or the like. Such disorders, syndromes, diseases, and problems will be apparent to those of ordinary skill in the art.

In an embodiment designed to be utilized by physicians, for example, medical illnesses, syndromes and diseases, such as those associated with Axis 3 635 may be solely or primarily assignable by a physician. In an embodiment, problems may be represented on less than all of the 5 axes (625-640 and Axis 5) and/or problems may be selected for less than all of the 5 axes.

In an alternate embodiment, illnesses, syndromes, diseases, disorders and/or the like may be represented in a variety of alternate ways. For example, the ICD-10 classification system may be utilized instead of the DSM-IV 5-axis classification depicted in FIG. 6. Still alternate and/or additional ways of displaying diagnoses may be used within the scope of the present disclosure.

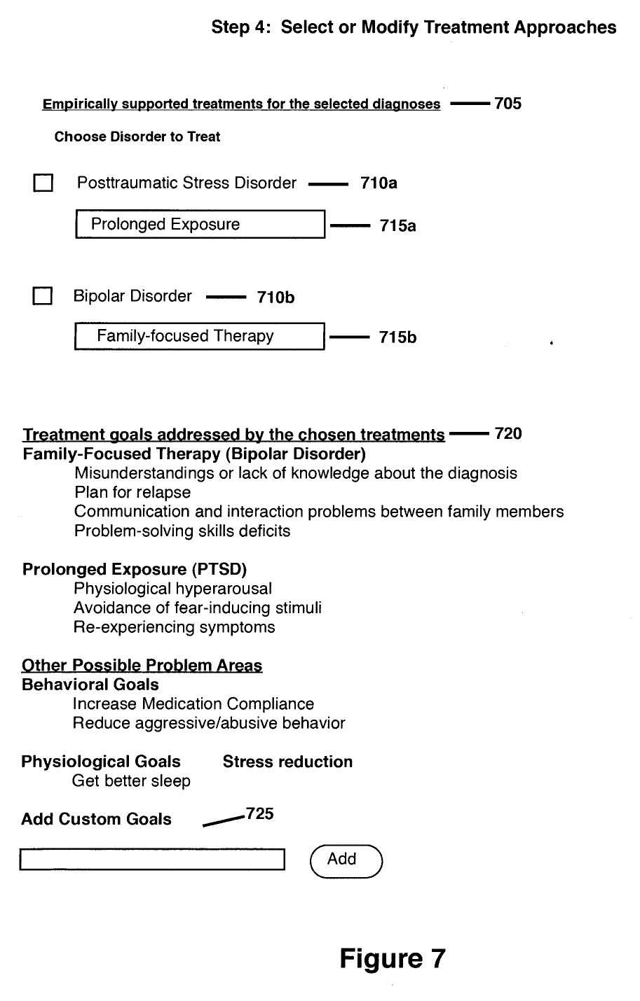

The treatment provider may then select 320 a treatment approach for the patient based on the one or more diagnoses. In an embodiment, the treatment approach may be dependent upon or result from the one or more selected diagnoses. FIG. 7 depicts an exemplary graphical user interface screen for selecting a treatment approach for a patient according to an embodiment. Alternative treatment approaches and goals may be provided to the treatment provider to assist in treating the patient. The treatment provider may select the treatment approach from these alternatives to treat each problem that the patient has. As shown in FIG. 7, the treatment graphical user interface may include a treatment approach section 705 that includes a list of one or more disorders, such as 710a-b, that are generated based on the diagnoses selected for the patient. In an alternate embodiment, the list of one or more disorders 710a-b may be generated based on automated decision trees, pattern matching algorithms and/or the like which cross-reference data from the initial clinical interview. In addition, the treatment approach section 705 may include a list of treatments, such as 715a-b, that are most appropriate for a given patient for each disorder or for any combination of disorders. In an embodiment, the list of treatments 715a-b for a particular disorder may be modified based on other diagnosed disorders or other information received from the initial clinical interview or during the course of treatment. For example, a list of treatments 715a for posttraumatic stress disorder may be automatically modified based on the diagnosis of bipolar disorder for the patient. Similar modifications may also be made based on, for example and without limitation, the gender of the patient, the medical history of the patient, and/or the like. In an embodiment, the list of treatments 715a-b may also be modified based on the credentials, preferences and/or the like of the treatment provider.

A treatment provider may select a disorder to treat and a treatment for such disorder from the list. In an embodiment, a treatment provider may opt to not treat each disorder at the same time. As such, less than all listed disorders may be selected for treatment by the treatment provider.

The treatment provider may also select 325 one or more treatment goals for the patient based on the treatment approach and various lifestyle, behavioral and/or psychological goals. As shown in FIG. 7, the treatment graphical user interface may further include a treatment goals section 720. The treatment goals section 720 may include a list of treatment goals (e.g., make more friends) and/or problem areas (e.g., alienates others) to be addressed during treatment. The treatment goals and/or problem areas may be organized into one or more categories. For example, goals and/or problems may be directed toward need areas in a patient's life that can be pursued in treatment, treatment approaches, disorders and/or the like selected for treatment by the treatment provider. In an embodiment, each listed goal or problem may be initially organized based on diagnosis, treatment approach, treatment goals, and/or the like so that a treatment provider does not inadvertently forget to assign a goal/problem. In addition, one or more custom goals and/or problems 725 may be entered by the treatment provider.

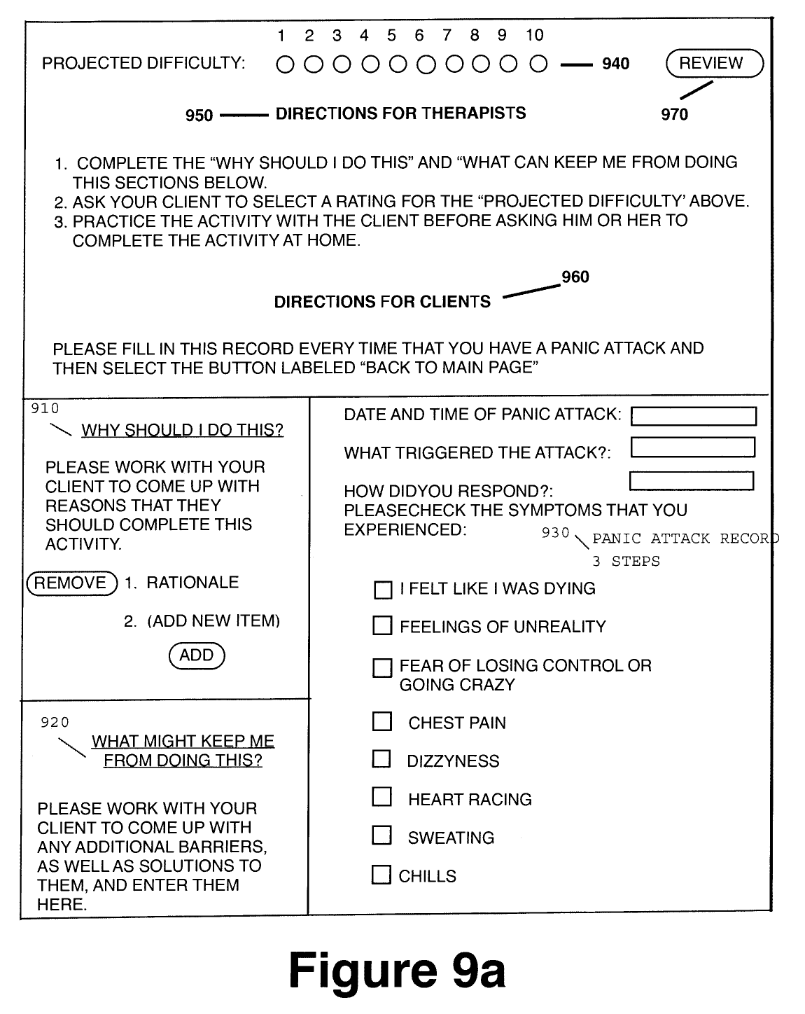

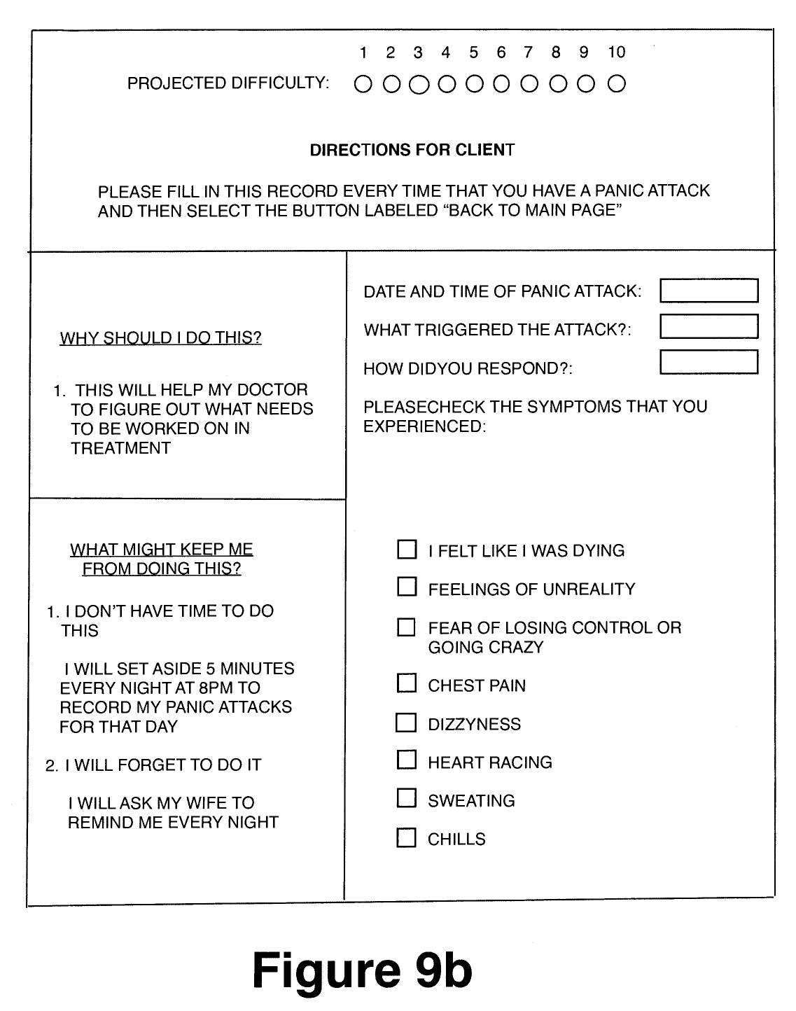

Once the goals and/or problems have been selected 325 for a patient, treatment for the patient may be initiated 330 that is coordinated with the diagnosis, treatment approaches, treatment goals/problems, and/or treatment provider's credentials identified by the above process. Information pertaining to the diagnosis, treatment approaches and treatment goals/problems may be displayed to treatment providers via a treatment provider version of the homework intervention graphical user interface screen, an embodiment of which is discussed below in reference to FIG. 9a.

A decision to modify or customize a homework intervention may be based on both objective criteria (e.g. the diagnosis, the treatment approach, and/or the goals of the treatment) and clinical judgment because many unquantifiable factors are considered by the treatment provider. With respect to the treatment rationale and barriers to treatment, factors to consider may include the patient's level of motivation to fully participate in treatment, family and friends' motivation to maintain the problem, the patient's interests (e.g., the patient may be motivated to go for a walk in the morning but not a swim at night), the patient's perceptions (e.g., the patient may perceive one activity as too time consuming but would be willing to engage in a separate activity), and/or the like. In addition, an intervention may be modified based on whether the treatment provider is able to provide a certain treatment (e.g., a particular treatment may require the treatment provider to be certified by a regulatory entity or the like).

Categories of differences in the directions provided for treatments may include the duration of the treatment, the intensity of the treatment (e.g., for cardiac aftercare, running or walking briskly or slowly may be potential alternatives based on the physical health of the patient), the manner in which the directions are framed (e.g., to suit the patient's belief systems and/or to encourage compliance), and/or the like. For example, if a parent believes in the maxim ‘spare the rod, spoil the child,’ telling the parent that spanking is wrong could lead the parent to remove the child from treatment.

Because the number of potential considerations are innumerable, it would be difficult to adequately account for such considerations and standardized treatments using actuarial methods, such as algorithms and decision trees. Accordingly, in an embodiment, the treatment provider may modify treatments by the system to accommodate the circumstances surrounding an individual patient. In this manner, interventions may be assigned to the patient to provide improved treatment over standardized treatment methods.

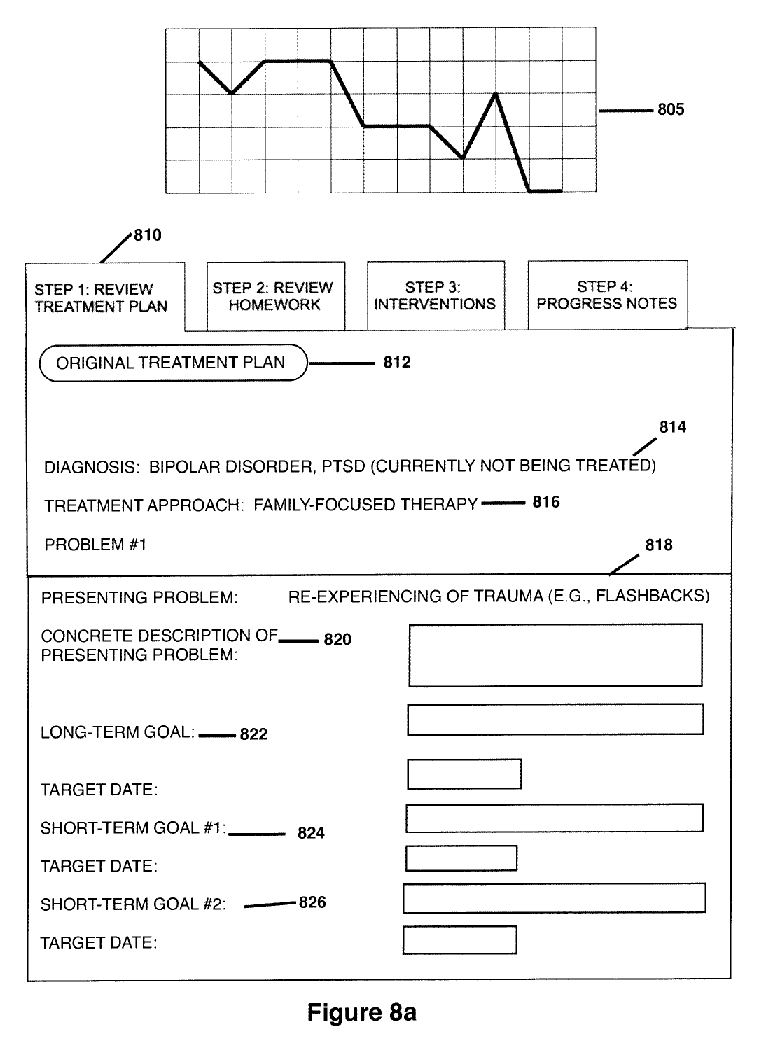

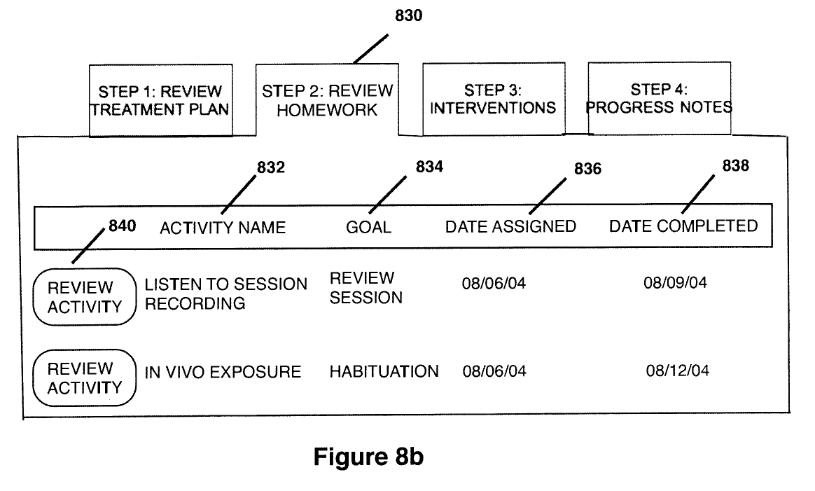

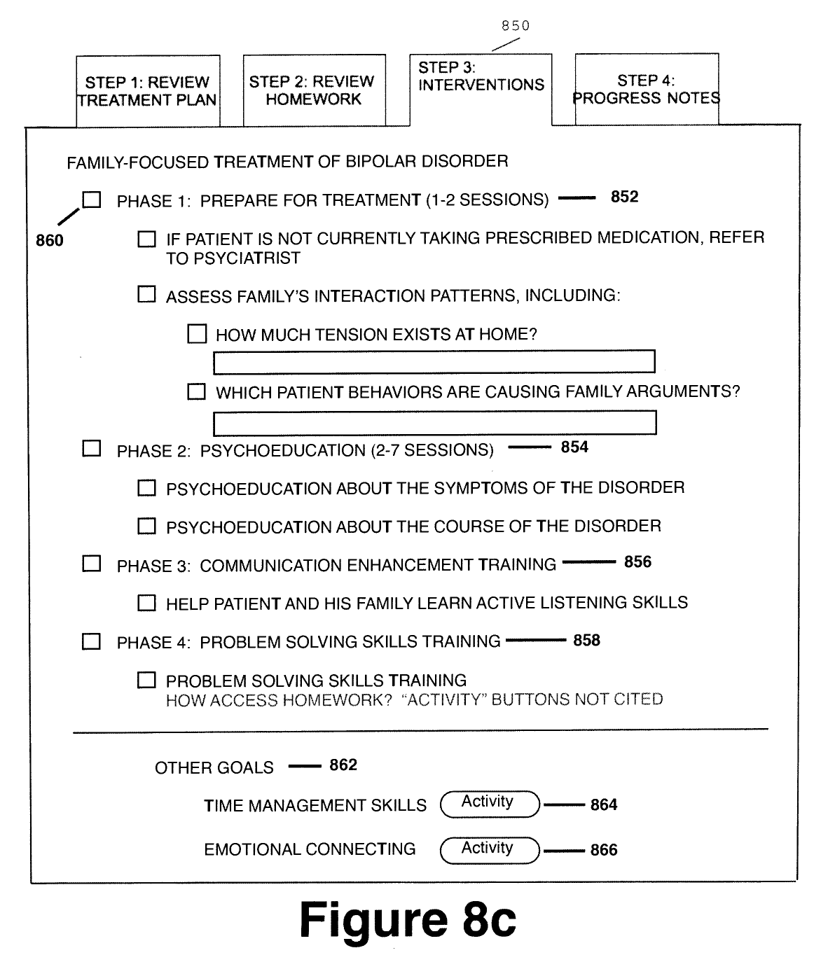

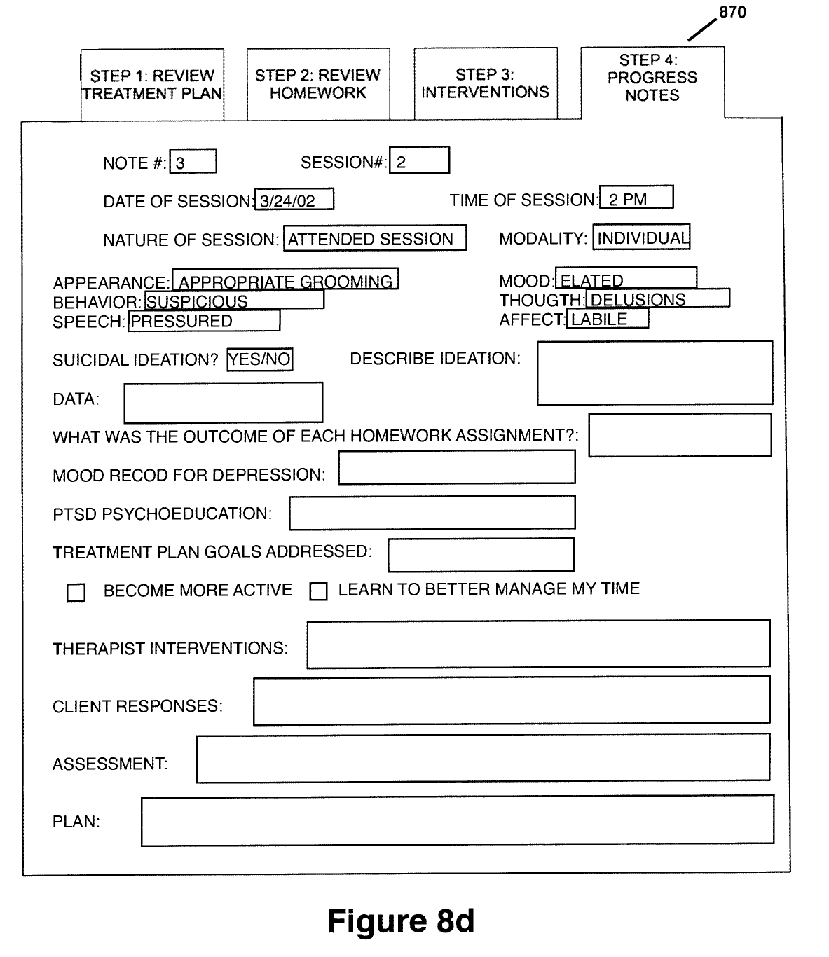

FIGS. 8a-8d depict exemplary graphical user interface screens for assisting a treatment provider with treating a patient according to an embodiment. As shown in FIG. 8a, the treatment provider, such as a therapist, may access a patient graphical user interface. In an embodiment, the patient graphical user interface may be the primary location through which treatment records for a patient are displayed and recorded with respect to the system. The patient graphical user interface may include one or more graphs and/or charts, such as 805, and one or more sub-sections designed to guide treatment providers through each treatment session for a given patient throughout the treatment process. Each graph, such as 805, may identify one or more recorded levels for a symptom over a time period or in relation to other symptoms or other factors such as behaviors, thoughts, environmental factors, and/or the like. The recorded level for a symptom may pertain to the prevalence of the symptom on a given day, at a given session and/or the like. Each value may be entered by either a treatment provider or the patient. A graph and/or a chart may be generated based on progress notes, homework interventions, biological sensors, in-session assignments, computer-generated data, such as time spent viewing audio-visual stimuli, word counts, data drawn from this and/or other electronic medical records, digital note pads, smart phones and/or the like.

The sub-sections can be navigated in any sequence. However, the sub-sections may be designated and ordered based on an anticipated sequence that would typically be followed during a treatment session, such as, for example, a cognitive-behavioral therapy session. Exemplary sub-sections may include, without limitation, “Review Treatment Plan,” “Review Homework,” “interventions,” “Progress Note,” and/or the like. Although the sub-sections are depicted as tabs in FIGS. 8a-8d, alternate ways of implementing the sub-sections for display to a user may be performed within the scope of this disclosure.

In an embodiment, a Review Treatment Plan tab 810 may be configured to display information regarding the treatment plan for a patient, as shown in FIG. 8a. A treatment provider may access the tab 810 to identify the core problems for the patient and the specific goals for the treatment process. As shown in FIG. 8a, the tab 810 may include a selectable icon 812 used to select among displaying the current treatment plan, displaying a previous treatment plan (if any), and constructing a new treatment plan. In an embodiment, a previous treatment plan may include a treatment plan performed by a previous treatment provider or the current treatment provider. The tab 810 may further display a list of the one or more diagnoses 814 pertaining to the patient, a list of the one or more treatment approaches 816 used by the treatment provider in providing treatment, and one or more charts, such as 818.

The one or more charts 818 may be automatically generated to correspond to a diagnoses, treatment approaches, goals, problems and/or the like associated with the patient. In an embodiment, a problem chart may be configured such that a treatment provider can submit the information from the problem chart to a governing board as part of an accreditation process. Each problem chart 818 may include, without limitation, a description of the problem 820, a long-term goal 822 and one or more short-term goals 824, 826. The treatment provider may provide a description of the problem 820 that specifically describes how a symptom manifests for the particular patient.

The long-term goal 822 may be automatically generated for the patient based on the identified problem 820 and/or other information. In an embodiment, the treatment provider may be enabled to modify the long-term goal 822 to address particular circumstances pertaining to the patient. One or more short-term goals, such as 824, 826, may also be provided in furtherance of the long-term goal 822. Each of the long-term goal 822 and the short term goals 824, 826 may include a target date by which the corresponding goal is intended to be accomplished.

In an embodiment, a Review Homework tab 830 may be configured to display information regarding homework activities for a patient, as shown in FIG. 8b. Each activity may include, for example and without limitation, an activity name 832, an activity goal 834, an assignment date 836 and a completion date 838. The activity name 832 may describe the type of activity to be performed. The activity goal 834 may describe the intended result of the treatment. The activity goal 834 for a particular activity may be, for example, a therapeutic goal or may simply serve to reinforce information previously provided to the patient (such as for an activity of reviewing a previous session). The assignment date 836 and completion date 838 for an activity may allow the treatment provider to determine whether the patient is performing their homework in a timely manner, among other things. Each activity listing may further permit the treatment provider to review the activity in more detail. For example, each activity may include a button or other icon 840 for initiating such a review, as shown in FIG. 8b.

In an embodiment, the Review Homework tab 830 may be accessed by the treatment provider in order to review and discuss homework activities and interventions. The Review Homework tab 830 may enable a treatment provider or other healthcare professional or supervisor to review all homework for a patient, whether or not the homework was attempted or completed. One or more graphs and/or charts may be provided to assist the treatment provider in the discussion with the patient. In an embodiment, the one or more graphs and/or charts may be auto-generated based on progress notes, homework interventions, biological sensors, in-session assignments, computer-generated data, such as time spent viewing audio-visual stimuli, word counts, data drawn from this and/or other electronic medical records, digital note pads, smart phones and/or the like. The graphs and/or charts may include data entered by the patient, a third party, a biological sensor, and/or the like during the course of the homework interventions that pertain to a particular area of interest, such as, without limitation, the patient's mood or emotional state when performing one or more activities. Comparison with previous time periods may also be performed within the scope of this disclosure to measure the patient's progress over a longer period of time, for example. Use of such data may be useful in providing feedback regarding the efficacy or effectiveness of the treatment to the patient.

In an embodiment, an Interventions tab 850 may be configured to display specific intervention plans, as shown in FIG. 8c. For example, FIG. 8c depicts an intervention plan for a patient with bipolar disorder. A specific intervention plan (i.e., treatment plan) may be automatically displayed to a treatment provider. An intervention plan may be generated based on statistical calculations, Monte Carlo simulations, other expert system-related processes, such as forward- and backward-chaining, decision trees, pattern recognition, algorithms and/or the like that consider information received during the setup process described above and/or information entered and/or generated during any other step of the treatment process. For example, the intervention plan may consider information received from the initial clinical interview, the diagnoses for the patient, the modality of treatment, the one or more disorders being treated, the one or more treatment approaches, problems that the patient is experiencing and/or the like.

As shown in FIG. 8c, the intervention plan may be generated and organized into phases, such as 852, 854, 856 and 858 as dictated by a treatment for a particular disorder. In an alternate embodiment, the intervention plan may be generated and organized as a session-to-session progression. Particular interventions may be included in each phase to assist the patient while progressing through the treatment process. Each phase description may include a check box, such as 860. After a treatment provider selects an “activity” button or other icon next to an intervention and the treatment provider, the patient and/or a third party completes the requirements associated with the intervention, the check box 860 may automatically be checked. In an embodiment, once all check boxes 860 within a phase are checked, the check box corresponding to the phase may automatically be checked and interventions within the next phase may become available. In an embodiment, requirements for a particular intervention may include customizing a homework intervention, assigning the intervention, reviewing the intervention in one or more subsequent sessions, entering data for an in-session intervention and/or the like. The patient may also be required to attempt or complete an intervention to a specified extent prior to a check box 860 being automatically checked.

In an alternate embodiment, a treatment provider may manually check a check box, such as 860. For example, the treatment provider may check a check box 860, for example and without limitation, when one or more requirements are determined to have been completed. In an embodiment, the check boxes 860 may be used to monitor activities being performed on behalf of a patient. For example, an insurance provider, regulatory entity, supervisor and/or other third party may monitor progress made with respect to a particular patient.

The Interventions tab 850 may further include additional goals under an “Other Goals” entry 862. Each additional goal may include an activity button or other icon, such as 864, which redirects the treatment provider to a separate page used to define an activity to achieve the goal. In addition, a button or other icon 866 may be used to select additional homework activities from a graphical user interface including a list of all activities selectable by a treatment provider using the system.

A treatment provider may assign and/or administer one of, for example, three general types of interventions using the system: a homework intervention, an in-session intervention, and an in-session intervention that includes one or more follow-up homework interventions. The interventions may be stored in the system and/or programmed by a particular treatment provider.

In an embodiment, each homework intervention may be viewed in one of three modes: treatment provider, patient, and review. FIG. 9a depicts an exemplary graphical user interface screen for specifying a homework intervention according to an embodiment. The graphical user interface screen depicted in FIG. 9a is intended to be used by a treatment provider to specify and/or customize a homework intervention for a patient. Modifications made to the treatment provider version of the homework intervention may affect the information displayed to the patient on a patient version of the homework intervention, such as is shown in FIG. 9b.

As shown in FIG. 9a, the treatment provider homework intervention screen may include a plurality of sections for providing information to the patient. In a rationale section 910, the treatment provider may add, delete and/or modify treatment rationales to explain to the patient why the homework intervention is necessary or beneficial. The treatment rationales may be used as points of discussion with the patient during a session and/or a reminder for the patient in an “at home” setting. In an obstacles section 920, the treatment provider may also list, add, delete, and/or modify one or more barriers to compliance (i.e., obstacle) for the patient. Each of these sections may be automatically loaded with information retrieved from a database. However, the treatment provider may modify each section to direct the treatment to the particular patient for whom it is intended. In an embodiment, the obstacles section 920 may further include one or more solutions for overcoming an obstacle. The treatment provider may add, delete and/or modify one or more solutions in the obstacles section 920.

A description of the steps to perform during the homework intervention may be included in a recording section 930. The recording section 930 may enable the patient to access other functions of the homework intervention, such as recorded audio-visual media, various selection icons and menus, and recording features that enable the patient to monitor and record symptoms, responses to the intervention, behaviors, thoughts, environmental influences, sleep patterns, drug use, alcohol use, prescription use and/or the like. A patient may enter data using, for example and without limitation, a drop down menu, radio buttons, a free text field, a biological sensor, a diagnostic instrument and/or the like.

A feedback request 940 may be specified to request feedback from the patient regarding the difficulty of the homework intervention or the perceived difficulty of the intervention prior to completion by the patient. Retrieving information from the patient regarding the predicted difficulty may assist the patient in analyzing whether their initial evaluation tends to be accurate, and, if not, may encourage the patient to try additional intervention tasks if the actual difficulty is less than the predicted difficulty.

Directions 950 for the treatment provider and directions 960 for the patient may also be included. The system may have pre-loaded directions 950 for the treatment provider that describe how to address the information presented on the screen to the patient, how to treat the patient, and how to amend the information to be presented to the patient during the home treatment. The treatment provider may amend the directions 960 for the patient and/or other sections of the homework intervention in concert with the patient in order to better ensure compliance by the patient and to increase the potential that the intervention will be effective.

A practice button or other icon, such as 970, may be selected to bring up a patient version of the homework intervention graphical user interface screen, such as in shown in

FIG. 9b. In an embodiment, the patient version of the homework intervention screen may be substantially similar to a corresponding treatment provider version of the screen, but without the ability to change, move or add one or more prompts for data on the screen. Presentation of the patient version of the screen may be used to familiarize the patient with the screen prior to conducting the homework intervention.

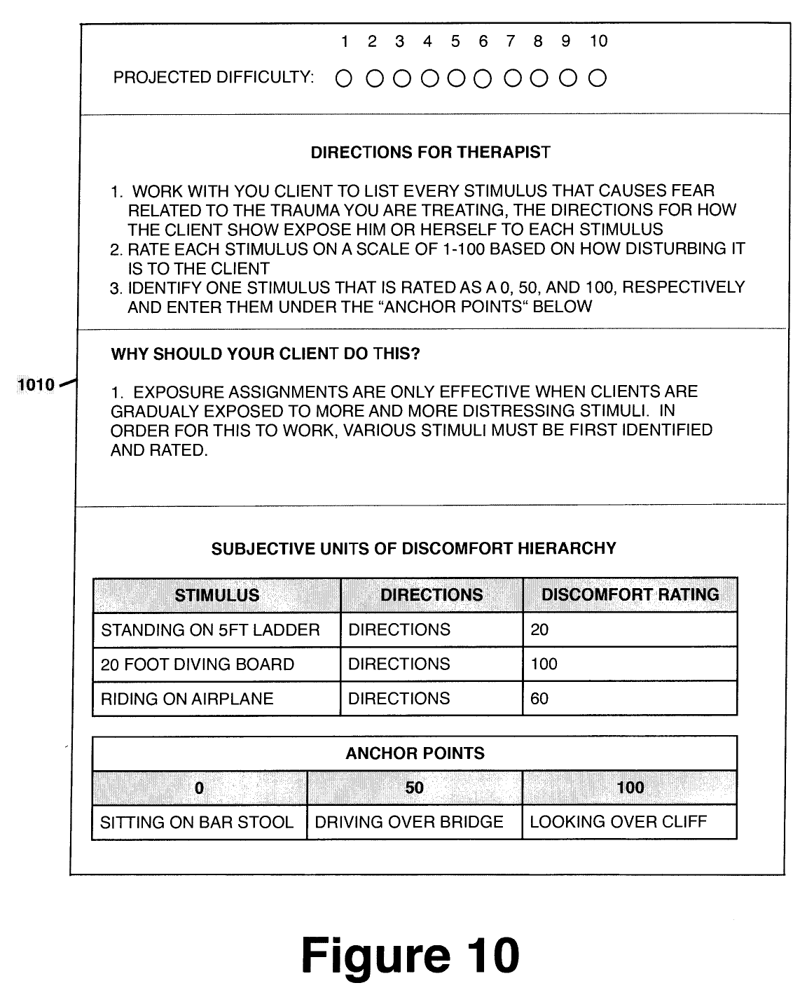

FIG. 10 depicts an exemplary graphical user interface screen for specifying and conducting an in-session intervention according to an embodiment. Because an in-session intervention is intended to be administered by the treatment provider, the screen may not include directions for the patient, a treatment rationale, an obstacles section as described above in reference to FIG. 9a and/or the like. As shown in FIG. 10, the screen may include one or more treatment rationales 1010 to assist the treatment provider in describing the purpose of the intervention to the patient, directions 1020 for the treatment provider to administer the intervention, and questions 1030 for which the treatment provider can provide answers via one or more text boxes and/or the like. Other information may also be presented to the treatment provider within the scope of this disclosure. In an embodiment, an in-session intervention screen may include an icon that redirects the treatment provider to a homework intervention designed as a follow-up for the in-session intervention.

Once a given intervention, either in-session and/or a treatment provider version of a homework intervention, has been completed, the intervention may be marked as complete with respect to the intervention plan described in reference to FIG. 8c and either an additional intervention can be performed or a session can be ended. In an embodiment, a patient, a third party, a treatment provider, a supervisor and/or another individual may mark the intervention as complete. In an alternate embodiment, the intervention may automatically be marked as complete. If the session is ended, the treatment provider may enter information in the Progress Note tab 870 depicted in FIG. 8d. The Progress Note tab 870 enables the treatment provider to seamlessly integrate treatment with treatment records. Information retrieved as a result of an intervention can be associated with a patient by clicking on one or more of the tabs when recording information in the Progress Note tab 870. In an embodiment, the treatment provider may manually enter information into the Progress Note tab 870 for one or more interventions. For example, a list of homework interventions initiated during or after the previous session may be automatically provided by the system. Alternately or additionally, a list of all interventions attempted during a current treatment session may be automatically provided. The treatment provider may enter information regarding such interventions, such as the patient's ability to perform the steps of the intervention, the patient's mood, appearance, and/or the like. By automatically providing such information, the treatment provider is less likely to forget to enter information regarding a particular intervention. Moreover, a permanent electronic medical record may be created. In an embodiment, the treatment provider is required to enter information for each intervention in order to ensure that the provider's thoughts are accurately recorded. In an embodiment, the treatment provider may add additional interventions and intervention information manually.

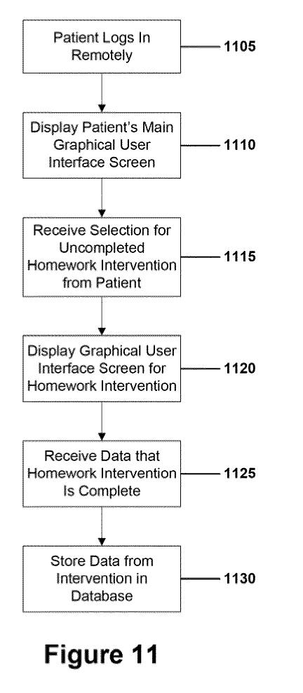

FIG. 11 depicts a flow diagram of an exemplary method of accessing a homework intervention by a patient according to an embodiment. As depicted in FIG. 11, the patient may remotely login 1105 to the system using the username and password assigned during the setup phase described above in reference to FIG. 3. In an embodiment, upon logging in 1105, the patient may be directed to the patient's main graphical user interface screen or a help page. In an embodiment, the patient may only be directed to the help page the first time that the patient logs in remotely (although the page may be accessed via a link accessible to the patient when accessing the system). Upon reviewing the help page, the patient may be directed to the patient's main graphical user interface screen.

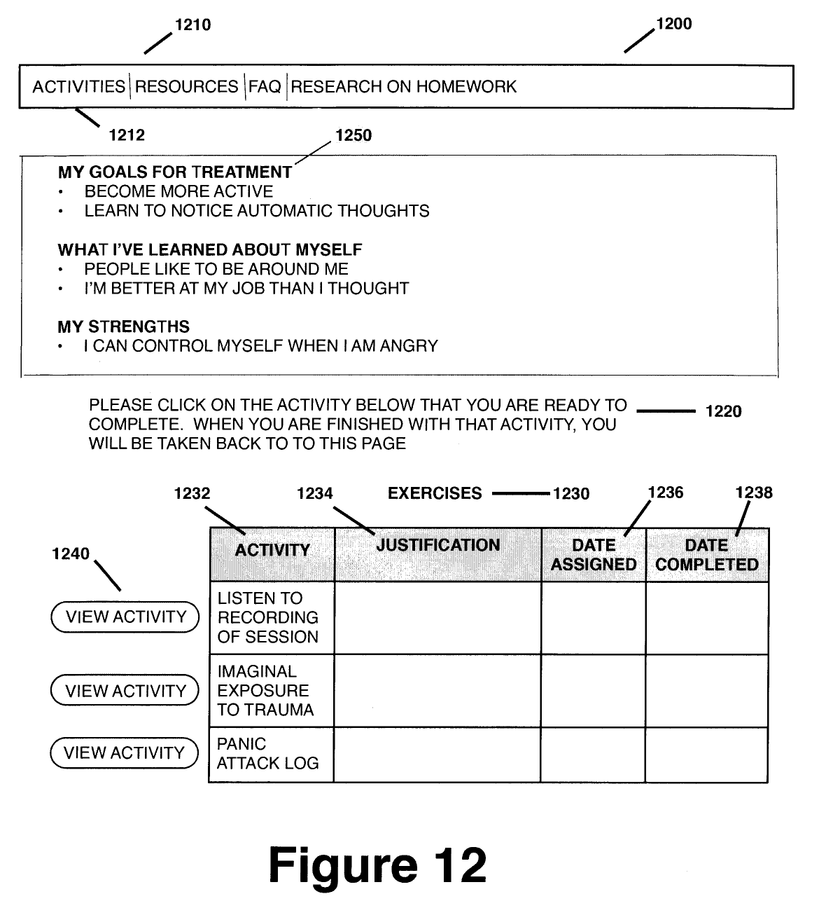

The patient's main graphical user interface screen, such as the screen depicted in FIG. 12, may be displayed 1110 to the patient. In an embodiment, the screen 1200 may include a link bar 1210, a directions section 1220, a homework intervention section 1230, a goals section 1250, and one or more other sections providing information to the patient. The link bar 1210 may include one or more selectable links or icons, such as an Activities link 1212, a Resource link 1214, a Frequently Asked Questions (FAQ) link 1216, and/or one or more other links.

The link bar 1210 may have persistent links or icons that are used to redirect the patient to specific pages from any page accessible by the patient. For example, the Activities link 1212 may be used to direct the patient to the patient's main graphical user interface screen 1200.

The directions section 1220 may include information designed to assist the patient in performing operations on a graphical user interface screen. For example, the directions section 1220 may describe navigation within a displayed graphical user interface screen or to an accessible graphical user interface screen.

The homework intervention section 1230 may include, for example and without limitation, a chart of all homework interventions (i.e., activities and/or exercises) assigned to the patient for a time period between sessions with a treatment provider. In an embodiment, the chart may include, without limitation, an activity name 1232, a justification 1234, a date assigned 1236, a date completed 1238, a view activity icon 1240. Additional and/or alternate information may be included within a chart within the scope of this disclosure.

The activity name 1232 may provide a brief description of the homework intervention. The justification 1234 may be a standardized description of the goal to be achieved as a result of performing the homework intervention and/or an explanation of why the intervention should not be aversive or harmful. The date assigned 1236 may identify the date on which the patient's treatment provider assigned the homework intervention to the patient, the date the patient opted to begin the homework intervention and/or the like. Similarly, the date completed 1238 may identify the date on which the patient completed the steps of the homework intervention, the date that the most recent entries were made and/or the like. If a particular homework intervention has not been completed, the date completed field 1238 for the homework intervention may be left blank or may have a designator requesting that the patient complete the homework intervention. In an embodiment, a homework intervention may have a plurality of entries in the date completed field 1238 corresponding to each successful completion of the homework intervention by the patient in a time frame. The view activity icon 1240 may direct the patient to a graphical user interface screen that more fully describes a corresponding homework intervention and allows the patient to attempt the intervention and/or review the outcome of a previously attempted or completed intervention/assignment.

The goals section 1250 may include, without limitation, text automatically generated by the system based on the long and short term goals entered by the treatment provider above. Other information such as “what I've learned about myself” and “my strengths” may display text entered by the treatment provider based on discussions with the patient during a session. The patient's main graphical user interface screen may be configured to display any

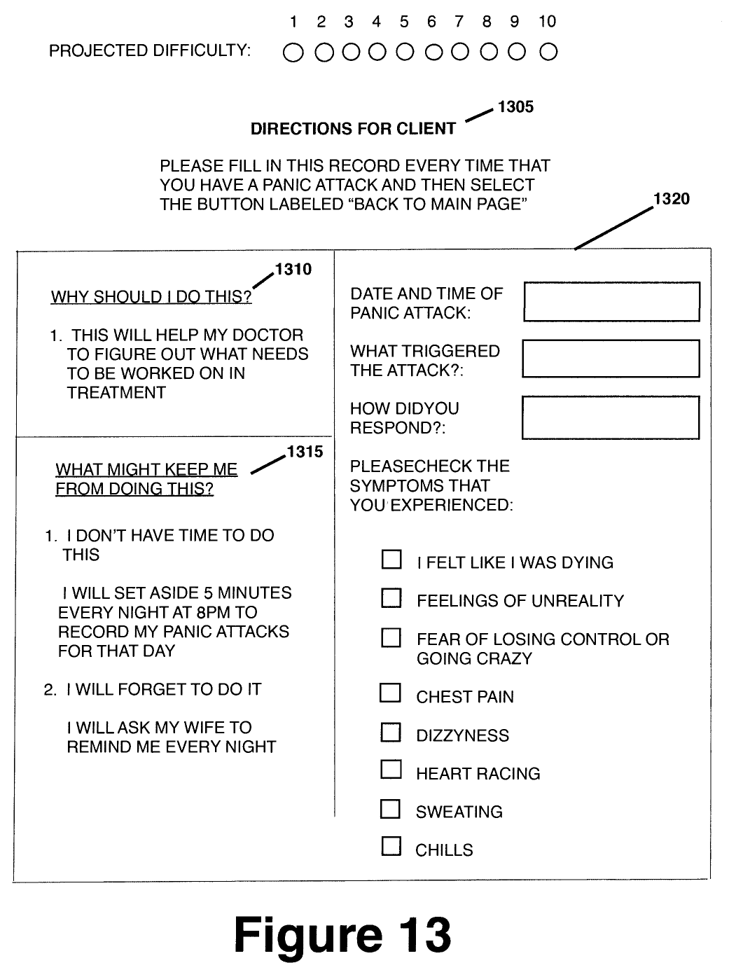

Referring back to FIG. 11, a selection for a view activity icon 1240 for a homework intervention that has yet to be completed may be received 1115 from the patient. Upon receipt of such a selection, the system may direct 1120 the patient to a patient graphical user interface screen for the homework intervention. As shown in FIG. 13, the patient version of the screen may provide directions 1305 for completing an intervention, one or more treatment rationales 1310, one or more potential obstacles and plans to overcome such obstacles 1315, and one or more questions 1320 to be answered by the patient. Each of the directions 1305, treatment rationale 1310, and obstacles 1315 sections may display data based on information input by the treatment provider. Answers to the questions 1320 may be supplied via text entry boxes, icons, menus and/or the like. The questions 1320 may be designed to allow the patient to self-monitor and record the outcome of, for example and without limitation, cognitive, behavioral and/or medical homework interventions.

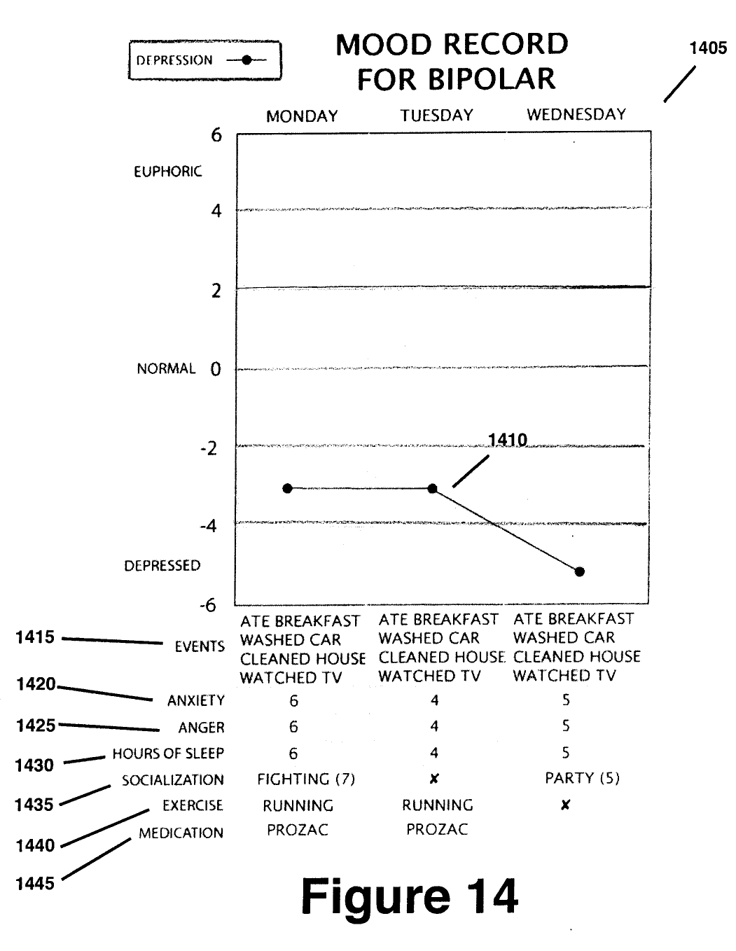

In an embodiment, the patient version of the graphical user interface screen for a homework intervention may include a difficulty rating to be assigned by the patient while practicing the activity with the treatment provider. The rating may allow the patient to make a prediction as to the difficulty of the behavior prior to attempting the intervention to predict the difficulty of the intervention. After completing the intervention, the patient may provide information as to how difficult the intervention was to complete. As such, the values may be compared to determine whether the patient is accurately assessing the difficulty of an intervention. In addition, the rating may assist the treatment provider in remembering to make sure that a particular intervention is not so difficult that the probability of completion is low or that the probability of failure is high, which could lead to increased feelings of hopelessness and resignation on the part of the patient. Information retrieved from the patient version of the graphical user interface screen may be used in the review version of the graphical user interface screen when the patient visits the treatment provider, described below in reference to FIG. 14.

In an embodiment, the patient version of the graphical user interface screen for a homework intervention may display one or more graphs and/or charts (not shown). A graph and/or chart may be automatically generated by the system and may include information entered by the patient, third parties, biological sensors (such as heart rate monitors, respiration monitors, sleep pattern monitors, and/or blood pressure sensors) and/or the like for one or more interventions.

The system may receive 1125 feedback information noting, for example and without limitation, that the intervention has not been attempted, has been attempted, has been partially completed and/or has been completed. The system may further receive 1125 feedback information pertaining to data entered by the patient and/or a third party in text fields, menu selections, when performing of the homework intervention. Additional and/or alternate feedback information, such as computer-generated data (such as time spent viewing audio-visual stimuli, word counts, data drawn from one or more electronic medical records, digital note pads, smart phones and/or the like), biological sensors, and/or the like, may also be received 1125 from a patient computer and/or mobile device. Feedback information from the homework interventions may be stored 1130 in a system database and may be reviewable using the review version of the graphical user interface screen, or may be accessed via the Review Homework tab 830 of the patient graphical user interface screen described in FIG. 8b.

In an embodiment, the feedback information may be used to automatically modify one or more interventions and/or to generate one or more new interventions for the patient. For example, if a patient is being treated for depression and the system interprets feedback information received from the patient as being indicative of worsening depression, a new or modified intervention may be generated for the patient in order to treat the depression. The new or modified intervention may be presented to the patient in a similar manner to that described above.

FIG. 14 depicts an exemplary graphical user interface screen used to review data recorded from one or more interventions according to an embodiment. As shown in FIG. 14, a graph and/or chart 1405 may be used to display information pertaining to a characteristic of interest obtained as a result of the patient's self assessment while conducting one or more homework interventions. For example, the graph 1405 may include an assessment of how depressed 1410 the patient is based on information provided by the patient in between sessions with the treatment provider. Alternately, a graph 1405 may depict the intensity and frequency of panic attacks for a patient or a patient's subjective and objective response to exercise after a heart bypass operation. In an embodiment, data for the assessment may be entered by the patient, a third party, retrieved from biological sensors, and/or the like. The patient may list one or more events 1415 that took place each day, provide ratings for how anxious 1420 and angry 1425 the patient felt and identify the number of hours of sleep 1430 the patient had on each day. In addition, other measures, such as types of socialization 1435 experienced by the patient, exercise 1440 undertaken by the patient, and types of medication 1445 taken by the patient during the day may be recorded. Other information may also be recorded with the graph within the scope of this disclosure. The treatment provider may use the information during a treatment session to detail aspects of the patient's behavior that exacerbate or ameliorate the patient's depression (or other condition) by reviewing such information with the patient. Similarly, the patient may review such information independently from the treatment provider to evaluate triggering events for undesirable behaviors, symptoms and/or the like.

Other groups of healthcare professionals may also access information contained within the system within the scope of this disclosure. For example, supervisors, researchers and collaborative care providers may have access to various information contained within the system.

A supervisor that manages one or more supervised treatment providers may be able to review records pertaining to patients treated by the supervised treatment providers. Supervised treatment providers may designate a supervisor during an account formation process. Alternately, the supervisor may create accounts for each supervised treatment provider.

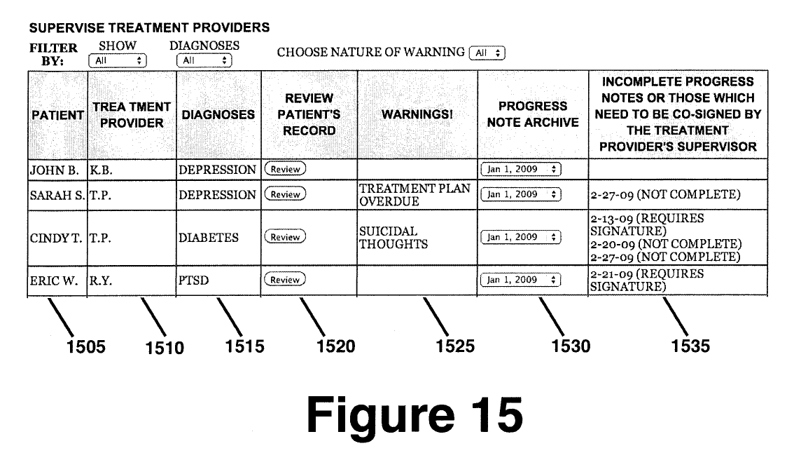

A supervisor may be able to access substantially the same pages as a treatment provider, as described above in reference to at least FIGS. 1, 2, 4-10 and 14. In addition, the supervisor may be able to access a supervisor graphical user interface screen, such as the one depicted in FIG. 15. As shown in FIG. 15, the supervisor graphical user interface screen may include a list of patients 1505 that are being treated by every treatment provider under the supervision of the supervisor. A designator identifying the treatment provider 1510 that is primarily responsible for treating the patient may be associated with each patient 1505. In addition, each patient record may include, without limitation, one or more associated diagnoses 1515, a link or icon directed to the patient's treatment plan 1520, a list of warnings 1525, if any, pertaining to the patient, a list of progress notes 1530 pertaining to the patient, and a list of progress notes 1535, if any, for which additional information is required. In an embodiment, some or all of the information in the chart may be automatically received from a database.

The one or more diagnoses .1515 associated with the patient may refer to at least one condition for which the patient is being treated by the treatment provider. The one or more diagnoses 1515 may be automatically received from a database based on information supplied by, for example, a treatment provider, a third party and/or the patient.

The icons directed to each patient's treatment plan 1520 may be used to redirect the treatment provider, when selected, to a treatment provider graphical user interface screen, such as the one shown in FIG. 8a, except that data may not be editable within such screen. In an embodiment, the supervisor may review the treatment plan by accessing such icon 1520. In an embodiment, data may not be modifiable via the supervisory graphical user interface screen. As such, all data in the patient's record may be modifiable solely by the patient and/or the treatment provider assigned to the patient. In an alternate embodiment, one or more third parties, such as a supervisor, may be able to modify data in a patient's record.

One or more warnings 1525 may be displayed in a supervisory record corresponding to a patient. The warnings 1525 may be designed to alert the supervisory treatment provider to an event that could compromise the effectiveness of the treatment, compliance with treatment standards or regulations, the safety of the patient or others and/or the like. A warning 1525 may be added to a patient record based on information retrieved from the initial clinical interview with the patient, information entered during a session with a treatment provider, information entered by the patient or a third party, such as therapeutic staff support, a collaborating physician and/or the like, information received from biological sensors, and/or the like. The warning 1525 may result from information received as a result of a homework intervention, via an interfaced electronic medical record, and/or the like. The warning 1525 may be linked to the patient record when information in the database meets or exceeds one or more criteria set by the supervisor, the system and/or a third party, such as a regulatory entity or third party payer.

In an embodiment, all progress notes 1530 pertaining to a patient may be listed, for example, in a drop-down menu and/or the like. A progress note 1530 may be referenced, for example, by the date on which it was entered into the system or the date of the treatment session to which it pertains. Selection of a progress note 1530 may display the information entered by the treatment provider on such date and/or other information.

Progress notes 1535 for which information is required may be separately listed. In an embodiment, progress notes 1535 that have not been approved or are deemed incomplete may be listed in separately. In an embodiment, a progress note 1535 may be required to be approved by the supervisor. The supervisor may provide approval by, for example, clicking an icon associated with the progress note 1535. In an embodiment, a progress note 1535 may be deemed incomplete if, for example, an in-session activity or homework activity is currently being performed or has not been performed or less than all required information is entered into a progress note. In an alternate embodiment, a progress note 1535 may be deemed incomplete if a practice management application (e.g., an application for billing and/or scheduling treatment) that is linked to, built into, or comprises a part of the system indicates that the patient attended a session or otherwise made contact with the treatment provider and a progress note has not been initiated.

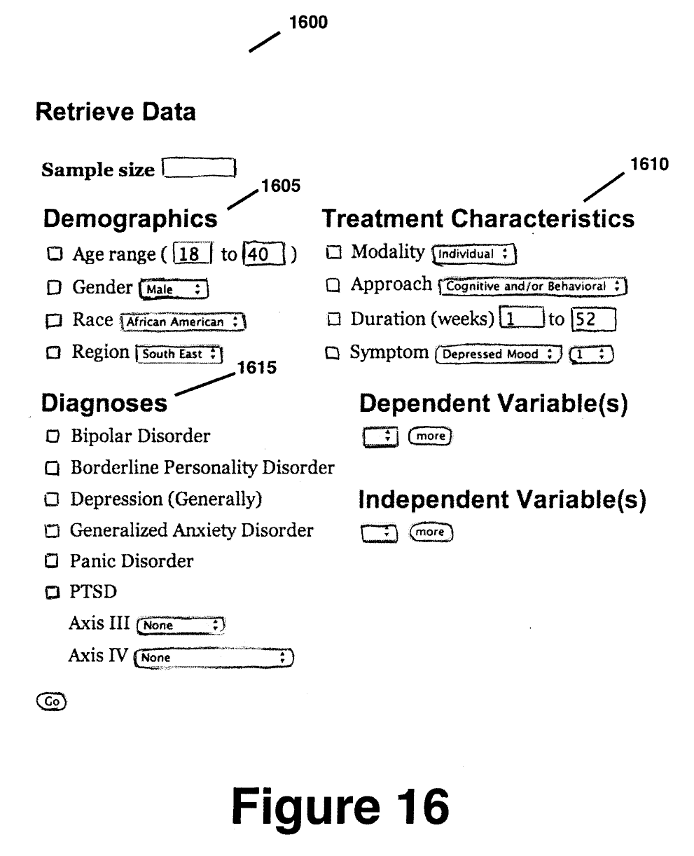

A researcher may be provided with access to pages assigned to one or more treatment providers in order to review records pertaining to a plurality of patients. The researcher may be able to access substantially the same pages and/or perform substantially the same functions as a treatment provider, as described above in reference to at least FIGS. 1, 2, 4-10 and 14, or a supervisor, as depicted in FIG. 15. In addition, the researcher may be able to access a researcher graphical user interface screen, such as the one depicted in FIG. 16.

As shown in FIG. 16, the researcher graphical user interface screen 1600 may enable the researcher to select a variety of demographic parameters 1605, treatment characteristic parameters 1610, diagnosis parameters 1615 and/or the like. The selectable demographic parameters 1605 may include, without limitation, an age or age range, a gender, a race, a region of the country in which the patient lives, works or is being treated, and/or the like. The treatment characteristic parameters 1610 may include, without limitation, a modality for the patient, a treatment approach, a duration of the treatment (e.g., in weeks), a symptom, and/or the like. The diagnosis parameters 1615 may include, without limitation, bipolar disorder, borderline personality disorder, depression, generalized anxiety disorder, panic disorder, post-traumatic stress disorder, physical disorders, illness, or diseases, psychosocial and environment problems, attention deficit hyperactivity disorder, oppositional defiant disorder, autism, Aspergers disorder, conduct disorder, reactive attachment disorder and/or the like. In an embodiment, the selected parameters may be considered disjunctively. In an alternate embodiment, the selected parameters may be considered conjunctively. In an alternate embodiment, the selected parameters may be considered disjunctively within a particular class of parameters, but conjunctively among classes of parameters.

The researcher graphical user interface screen 1600 may further include one or more dependent variables and one or more independent variables. An independent variable is a variable that is being manipulated or changed in a study. A dependent variable is an observed result of the independent variable being manipulated. Selectable dependent variables and independent variables may be listed, for example and without limitation, in drop down menus, check box menus and/or the like.

In an embodiment, independent variables may include, without limitation, all of the treatment characteristic variables 1610, how long the patient stayed in treatment, satisfaction with treatment, and the like. In an embodiment, dependent variables may include, without limitation, the extent to which goals were achieved; whether or not the patient continued or dropped out of treatment (this could also be an independent variable, with, as an example, an dependent variable of symptom change); a patient's overall satisfaction with treatment; a homework completion rate (for example, effected by an independent variable such as treatment approach) and/or the like. In an embodiment, homework completion rate may also be an independent variable effecting a dependent variable, such as a symptom change.

When all parameters and variables have been selected, the researcher may submit the information to the central database. A file may be downloaded to the researcher's local computer system including, for example, a comma-separated value (CSV) format file of all of the data matching the parameters. In an embodiment, the data may include the entirety of the data for each patient other than patient-identifying information. In an embodiment, the data may include data from the initial patient interview, progress notes, homework or in-session interventions, and/or the like.

In an embodiment, a treatment provider may provide access to one or more patient records to one or more second treatment providers in order to provide collaborative care to the patient. In an embodiment, a therapist may provide access to a physician so that the physician can enter information into or view a patient's record to generate a graph, chart and/or the like that provides the system with information intended to effect the construction of an intervention plan, progress notes, warnings, data supplied to third party payers and/or regulatory entities, and/or the like. In an embodiment, a therapist may provide access to a physician so that the physician can enter information into or view a patient's record to determine an extent to which a given medication is helping or hindering treatment and/or the like. Conversely, the therapist may review medications or treatment regimens prescribed by the physician when creating a treatment plan for the patient. Other means for providing collaborative care to a patient may be performed within the scope of this disclosure.

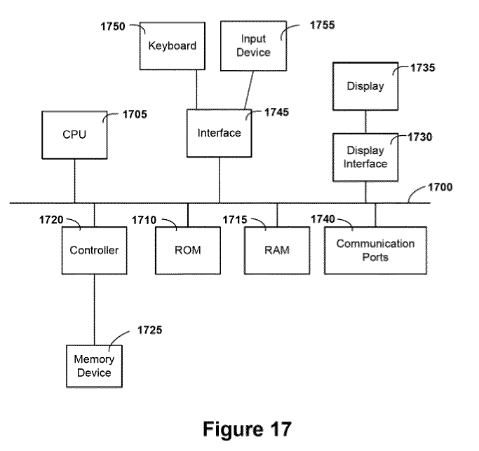

FIG. 17 depicts a block diagram of exemplary internal hardware that may be used to contain or implement program instructions according to an embodiment. As shown in FIG. 17, a bus 1700 serves as the main information highway interconnecting the other illustrated components of the hardware. CPU 1705 is the central processing unit of the system, performing calculations and logic operations required to execute a program. Read only memory (ROM) 1710 and random access memory (RAM) 1715 constitute exemplary memory devices.

A controller 1720 interfaces with one or more optional memory devices 1725 to the system bus 1700. These memory devices 1725 may include, for example, an external or internal DVD drive, a CD ROM drive, a hard drive, flash memory, a USB drive and/or the like. As indicated previously, these various drives and controllers are optional devices.

Program instructions may be stored in the ROM 1710 and/or the RAM 1715. Optionally, program instructions may be stored on a tangible processor-readable storage medium such as a compact disk, a digital disk, flash memory, a memory card, a USB drive, an optical disc storage medium, such as Blu-ray™ disc, and/or other tangible recording medium.

An optional display interface 1730 may permit information from the bus 1700 to be displayed on the display 1735 in audio, visual, graphic or alphanumeric format. Communication with external devices may occur using various communication interfaces 1740. An exemplary communication interface 1740 may be attached to a communications network, such as the Internet or an intranet.

The hardware may also include an interface 1745 which allows for receipt of data from input devices such as a keyboard 1750 or other input device 1755 such as a mouse, a joystick, a touch screen, a remote control, a pointing device, a video input device and/or an audio input device.

An embedded system may optionally be used to perform one, some or all of the operations described herein. Likewise, a multiprocessor system may optionally be used to perform one, some or all of the operations described herein.

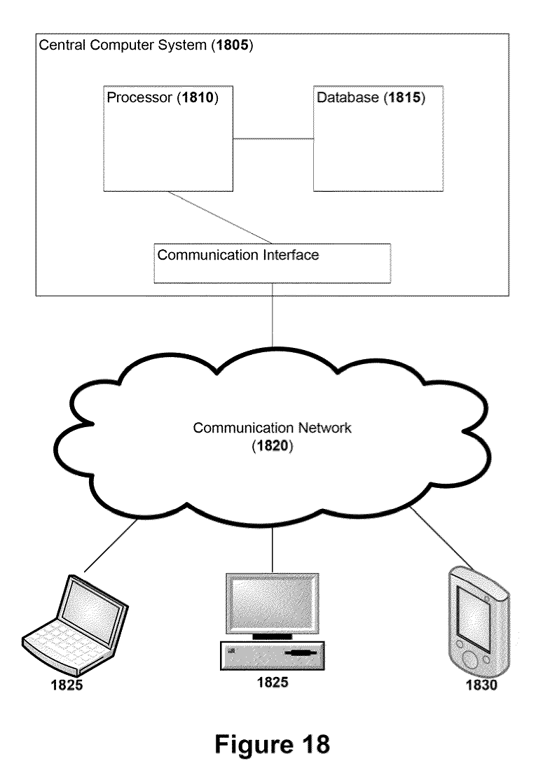

FIG. 18 depicts a block diagram of an exemplary computer network according to an embodiment. As shown in FIG. 18, a computer network may include a central computer system 1805, having one or more processors 1810, for implementing a database storage and retrieval system 1815, a communication network 1820 and one or more remote computer systems, such as 1825. In an embodiment, the central computer system 1805 may be operated under the direction of a treatment provider. In an alternate embodiment, the central computer system 1805 may be operated by a third party. Information may be entered into the system by a treatment provider, either directly into the central computer system 1805 or via a remote computer system 1825, or a patient, via a remote computer system. If information is entered via a remote computer system 1825, the information may be transmitted over the communication network 1820 to the central computer system 1805. The communication network 1820 may include one or more local area networks, such as an intranet, and/or one or more wide area networks, such as the Internet. The communication network 1820 may include wired and/or wireless networks. When the information is received by the central computer system 1805, a server 1810 may store the information in a database 1815 located in a storage medium. The database may associate the information with the treatment provider and/or the patient for whom it was entered.

In an embodiment, information may alternately be received from a wireless device 1830, such as an iPhone® from Apple Inc., a Blackberry® from Research in Motion Limited, a cellular phone, a personal digital assistant, and/or the like. Alternate mechanisms for connecting to the communication network 1820 will be apparent to those of ordinary skill in the art based on this disclosure.

In an embodiment, information may also be retrieved from the database 1815 by a remote computer system 1825, a wireless device 1830, or the central computer system 1805. Information may be retrieved in either a push or pull fashion. For example, a reminder may be transmitted to a patient via a wireless device 1830 to attempt a homework intervention. Alternately, a treatment provider may request information regarding a patient's progress from the central database 1815 in advance of a treatment session. information may be provided at a plurality of other times and for a plurality of other reasons within the scope of this disclosure.

Alternate methods of using the system may be performed based on the teachings disclosed above. For example, school-based intervention systems and methods may be developed according to the principles and teachings of this disclosure. In such an embodiment, guidance counselors, teachers and/or other school personnel may utilize such a system to provide treatment for behavioral problems effecting the school environment, and/or the academic success of a student. The system may also be used to provide direction for parents of students having a behavioral or academic issue. For example, the system may be used to assist in the provision of treatment for attention deficit hyperactivity disorder, oppositional defiant disorder, autism, aspergers disorder, oppositional behavior, conflict with other students, truancy, frequent tardiness, attention problems or disorders, poor study skills and/or habits, failure to complete and/or turn in work, and/or the like.

One or more differences may exist between an embodiment directed towards a scholastic environment and the embodiments described above. For example, the treatment provider in the scholastic environment may not be licensed to provide medical or psychological treatment or work under the direction of a licensed individual, but may instead be a teacher, guidance counselor and/or the like. Moreover, the setup required to address a behavioral issue may require only a subset of the steps described above. For example, the setup in the scholastic environment may merely require performing one or more of the following operations: creating an account for the student, diagnosing the student's needs, and choosing one or more goals to accomplish and/or interventions. Additional and/or alternate operations may be performed.

In an embodiment, the person performing the homework intervention may differ from the person receiving instructions from the school personnel. In an embodiment, a parent, guardian, sibling, or other third party may access the account and administer the intervention. Exemplary interventions may include, without limitation, changing disciplinary practices, visually monitoring the student as scholastic homework is performed, helping the student manage time, speaking respectfully to the student, spending time with the student, teaching conflict resolution skills to the student, and/or the like. The third party may access the account and administer the intervention because the student may not be developmentally, intellectually, and/or emotionally effective at understanding and implementing such interventions.

In an embodiment, compliance may be monitored by one or more school personnel. Collaboration may be performed between one or more third parties, teachers, guidance counselors, therapists, physicians and/or the like with respect to the student. In an embodiment, the account may enable the school personnel, parents, guardians, teachers and/or the like to monitor academic homework compliance and/or grades by interfacing with one or more computer applications used to administer or record information, such as grades, pertaining to academic homework.

In an embodiment, a parent, guardian, sibling or other third party may have an account that displays intervention information for a plurality of students. In an embodiment, the parent, guardian, sibling or other third party may encourage and/or assist each student in complying with the directions provided by the school personnel.

In an alternate embodiment, the methods and systems described herein may be used to perform collaborative care, such as behavioral health rehabilitation services (i.e., wraparound treatment). Wraparound treatment may apply to multiple treatment providers and/or third parties treating a single patient. For example, wraparound treatment may be used to treat autism, Aspergers disorder, conduct disorder, oppositional defiant disorder, attention deficit hyperactivity disorder, reactive attachment disorder and/or the like.

In an embodiment, information pertaining to the patient entered by any treatment provider, third party and/or the patient may be available to each treatment provider when accessing the system. Wraparound services may be provided directly to a patient by a plurality of treatment providers, such as a therapeutic staff support, a therapeutic staff support assistant, a behavior specialist consultant and/or a mobile therapist.

In an embodiment, the behavior specialist consultant and/or the mobile therapist may perform the setup process. The behavior specialist consultant and/or the mobile therapist may also act as a supervisor for other treatment providers with respect to the patient. One or more treatment providers may have access to the system to enter data pertaining to the patient. An intervention may be accessed and administered to the patient by the therapeutic staff support and/or the therapeutic staff support assistant through a treatment provider graphical user interface disclosing intervention information, such as the screen shown in FIG. 8c, and/or the patient graphical user interface for homework intervention, such as the screen shown in FIG. 13. In an embodiment, a parent, guardian, sibling and/or other third party may also access and administer a homework intervention to the patient using the patient graphical user interface for homework intervention.

Using the techniques, systems and methods described in the present disclosure may result in increased accuracy of information stored in electronic medical records. Rather than depending upon dated information, the present disclosure teaches real time recording of pertinent data resulting in more accurate records. As such, collaborative care, among other types of care, may be more effective as each treatment provider may rely on more accurate and more timely information from other treatment providers, the patient and/or other third parties than would otherwise be available.

It will be appreciated that the above-disclosed and other features and functions, or alternatives thereof, may be desirably combined into many other different systems or methods. In addition, various presently unforeseen or unanticipated alternatives, modifications, variations or improvements may be subsequently made by those skilled in the art. Such systems, methods, alternatives, modifications, variations and improvements are intended to be encompassed within the scope of the present disclosure and by the following claims.

What is claimed is:

1. A computer-implemented system for managing treatment for a patient, the system comprising:
a processor;
a processor-readable storage medium in communication with the processor; and
a communication interface in communication with the processor,
wherein the processor-readable storage medium contains one or more programming instructions for performing the following:
receiving, by the processor via the communication interface, data pertaining to an intervention comprising one or more of a homework intervention, an in-session intervention, and a combination intervention,
storing the data pertaining to the intervention,
transmitting, via the communication interface, at least a portion of the data pertaining to the intervention to a patient computer,
receiving feedback information from the patient computer via the communication interface,
storing the feedback information in association with the data pertaining to the intervention, and
causing, via the processor, the feedback information to be transmitted to a treatment provider computer.

2. The system of claim 1, wherein the processor-readable storage medium further contains one or more programming instructions for performing the following:
receiving the feedback information from a biological sensor; and
transmitting the feedback information to the patient computer.

3. The system of claim 1, wherein the data pertaining to an intervention comprises one or more operations for a patient to perform in order to complete the intervention.

4. The system of claim 1, wherein the data pertaining to an intervention comprises one or more of the following: a goal, an obstacle to the goal, a proposed solution for overcoming the obstacle, and a prompt configured to elicit recording of data.

5. The system of claim 1, wherein the patient computer comprises a wireless device.

6. The system of claim 1, wherein the patient computer comprises a wired computing device.

7. The system of claim 1, wherein the one or more programming instructions for receiving data pertaining to an intervention comprises one or more programming instructions for receiving data pertaining to an intervention from the treatment provider computer.

8. The system of claim 1, wherein the treatment provider computer comprises a wireless device.

9. The system of claim 1, wherein the treatment provider computer comprises a wired computing device.

10. A method of managing treatment for a patient, the method comprising:
receiving, by a processor, data pertaining to a first intervention comprising one or more of a homework intervention, an in-session intervention, and a combination intervention;
storing the data pertaining to the first intervention in a database;
transmitting, via the processor, at least a portion of the data pertaining to the first intervention to a patient computer;
receiving, by the processor, feedback information from the patient computer;
storing the feedback information in the database in association with the data pertaining to the first intervention; and
displaying the feedback information to a treatment provider.

11. The method of claim 10, further comprising:
receiving the feedback information from a biological sensor; and
transmitting the feedback information to the patient computer.

12. The method of claim 10, further comprising:
automatically modifying, via the processor, a second intervention based on the feedback information received in the first intervention;
transmitting, via the processor, at least a portion of the data pertaining to the second intervention to the patient computer;
receiving, by the processor, second feedback information from the patient computer;
storing the second feedback information in the database in association with the data pertaining to the second intervention; and
displaying the second feedback information to the treatment provider.

13. The method of claim 10, wherein the data pertaining to an intervention comprises one or more operations for a patient to perform in order to complete the intervention.

14. The method of claim 10, further comprising:
receiving a modification to the data pertaining to the intervention, wherein the modification is based on one or more of a preference of a patient, a physical condition of a patient, a preference of a third party, and a level of certification of a treatment provider.

15. The method of claim 14, wherein the modification comprises a modification to one or more of a duration of the treatment, an intensity of the treatment, and a manner in which directions are provided to the patient.

16. The method of claim 10, wherein the data pertaining to an intervention comprises one or more of the following: a goal, an obstacle to the goal, a proposed solution for overcoming the obstacle, and a prompt configured to elicit recording of data.

17. The method of claim 10, wherein the patient computer comprises a wireless device.

18. The method of claim 10, wherein the patient computer comprises a wired computing device.

19. The method of claim 10, wherein the intervention comprises a treatment for one or more of a mental health condition, a behavioral health condition, and a medical condition.

20. A method of managing collaborative treatment for a patient, the method comprising:
receiving, by a processor, data pertaining to a first intervention comprising one or more of a homework intervention, an in-session intervention, and a combination intervention;
storing the data pertaining to the first intervention in a database;
transmitting, via the processor, at least a portion of the data pertaining to the first intervention to a patient computer;
receiving, by the processor, feedback information from the patient computer;
storing the feedback information in the database in association with the data pertaining to the first intervention; and
enabling access to the data pertaining to the first intervention and the feedback information to a plurality of treatment providers.

21. The method of claim 20, further comprising:
receiving the feedback information from a biological sensor; and
transmitting the feedback information to the patient computer.

22. The method of claim 20, further comprising:
automatically modifying, via the processor, a second intervention based on the feedback information received in the first intervention;
transmitting, via the processor, at least a portion of the data pertaining to the second intervention to the patient computer;
receiving, by the processor, second feedback information from the patient computer;
storing the second feedback information in the database in association with the data pertaining to the second intervention; and
enabling access to the data pertaining to the second intervention and the second feedback information to the plurality of treatment providers.
